(19) United States
(12) Patent Application Publication
(10) Pub. No.: US 2006/0188564 A1
(43) Pub. Date: Aug. 24, 2006

(54) Cellulose hard capsule enhancing mechanical film strength

(76) Inventor: Joo Hwan Yang, Bucheon (KR)

Correspondence Address: JACOBSON HOLMAN PLLC, 400 SEVENTH STREET N.W., SUITE 600, WASHINGTON, DC, 20004, US

(21) Appl. No.: 11/136,462

(22) Filed: May 25, 2005

(30) Foreign Application Priority Data
Feb. 21, 2005 (KR) 10-2005-14135

(51) Int. Cl. A61K 9/48; B29C 39/10

(52) U.S. Cl. 424/451; 264/4

(57) ABSTRACT

This invention provides a process for preparing a cellulose capsule enhancing mechanical film strength comprising the steps of: i) preparing a mixed solution of iota-carageenan and agar; ii) adding said mixed solution to an aqueous solution of solubilized cellulose; iii) adding sucrose fatty acid ester, potassium pyrophosphate and glacial acetic acid to the resulting admixutre; and iv) allowing obtained product to stand, adjusting its viscosity and forming a capsule from it.

### BACKGROUND OF THE INVENTION

The present invention relates to a cellulose hard capsule enhancing mechanical film strength prepared by adding specific polysaccharide gelling agent to cellulose base material and its preparation method. More particularly, this invention relates to a cellulose hard capsule enhancing mechanical film strength prepared by the steps comprising i) adding sucrose fatty acid ester, potassium pyrophosphate and glacial acetic acid to cellulose base material solution; ii) adding mixed solution of iota-carageenan and agar as gelling agent to obtained mixture; and iii) allowing obtained product to stand, adjusting its viscosity and forming a capsule from it.

The traditional material for forming the capsule is gelatin, because it has the correct and quite ideal properties. Nevertheless, gelatin has some disadvantages which make it necessary to have other capsule materials available. A major unfavorable aspect is the animal origin of gelatin. Other disadvantages are the inconveniences of relatively high water content (10˜16%) and the loss of elasticity with decreasing water content. Furthermore gelatin capsules are sensitive to heat and humidity which affects the usability of the product.

As a gelatin substitute, cellulose hard capsule has been disclosed. Cellulose hard capsule is proper to insert hygroscopic material because the water content of cellulose hard capsule is less than 7% which is lower than that of gelatin capsule. Further, there is no problem of protein denaturation raised in gelatin because cellulose is originated from plant. Of course, it is useful to vegetarian.

However, there are some handicaps in cellulose hard capsule, because gelation of cellulose solution is not sufficient which results in low film strength and low stability of cellulose hard capsule. To improve the gelation of cellulose solution, methods for adding various polysccharides in cellulose solution have been disclosed.

In U.S. Pat. No. 5,431,917, cellulose hard capsule for pharmaceutical drugs was disclosed. In the examples of this disclosure, kappa-carageenan was disclosed as preferred gelatinizing agent and potassium chloride was disclosed as preferred auxiliary for gelation.

On the other hand, in U.S. Pat. No. 6,517,865 B2, a capsule film composition comprising i) 90˜99.98 wt % of at least one cellulose ether, ii) 0.01˜5 wt % of gellan gum, and iii) 0.01˜8 wt % of sequestering agent selected from ethylene diamine tetraacetic acid (EDTA), sodium citrate, citric acid or combination thereof was disclosed.

Further, in U.S. Pat. No. 6,410,050 B1, the inventor himself also disclosed a cellulose hard capsule prepared by the process comprising i) preparing a mixed solution of pectin and glycerin; ii) adding said mixed solution to solubilized cellulose aqueous solution; iii) adding glacial acetic acid, calcium gluconate and sucrose fatty acid ester to said mixture; and iv) allowing obtained product to stand, adjusting its viscosity and forming a capsule.

However, all cellulose hard capsules prepared by above disclosed methods have drawbacks of low mechanical film strength and low oxygen transparency compared to those of conventional gelatin hard capsule.

The reason why cellulose hard capsule has low mechanical film strength is caused by the physical property of cellulose. Therefore, the following problems occur at the time of preparing the cellulose hard capsule and filling the drug or food in this capsule.

First, bad joint occurs when the film of cap and the film of body separately formed are jointed at the joiner block because of low mechanical film strength of cellulose film.

Second, mashed badness occurs when capsules are transferred among drums in capsule inspection machine, capsule printing machine and/or capsule filling machine because of low mechanical film strength of cellulose film.

Third, mashed badness occurs when capsules are pushed by rubber roll in order to transcript the ink from rubber roll to surface of capsule during the spin printing because of low mechanical film strength of cellulose film.

Fourth, telescope badness occurs when cap and body of capsule are jointed after filling the food or drug in capsule during the filling works because of low mechanical film strength of cellulose film.

Therefore, the enhancement of film strength is required for commercial use of cellulose hard capsule. The present invention accomplished the enhancement of film strength of cellulose hard capsule by adding specific polysaccharide gelling agent to cellulose base material. Further, the present invention developed cellulose hard capsule enhancing film strength, which also reduces bad joint, mashed badness and/or telescope during the preparation of cellulose hard capsule.

### SUMMARY OF THE INVENTION

The object of the present invention is to provide a process for preparing a cellulose capsule enhancing mechanical film strength comprising the steps of: i) preparing an aqueous solution of solubilized cellulose; ii) adding sucrose fatty acid ester, potassium pyrophosphate and glacial acetic acid to an aqueous solution of solubilized cellulose; iii) adding a mixed solution of iota-carageenan and agar to the resulting admixutre; and iv) allowing obtained product to stand, adjusting its viscosity and forming a capsule from it.

Further, the present invention provides a process for preparing a cellulose capsule enhancing mechanical film strength according to claim 1, comprising the steps of: i) preparing 100 wt part of an aqueous solution containing 18˜21 wt part of solubilized cellulose; ii) adding 0.1˜0.5 wt part of sucrose fatty acid ester, 0.05˜0.3 wt part of potassium pyrophosphate and 0.01˜0.2 wt part of glacial acetic acid to 100 wt part of an aqueous solution of solubilized cellulose; iii) adding a mixed solution of 0.1˜1.0 wt part of iota-carageenan and 0.02˜0.5 wt part of agar to the resulting admixutre; and iv) allowing obtained product to stand, adjusting its viscosity and forming a capsule from it.

The weight ratio of iota-carageenan and agar is preferably 3.5˜4.5:1 and said cellulose is preferably hydroxypropylmethylcellulose (HPMC).

Said glacial acetic acid is added to neutralize the pH of an alkaline solution of solubilized cellulose.

On the other hand, the present invention also provides a cellulose hard capsule prepared by above preparation method comprising 100 wt part of cellulose, 0.5˜2.5 wt part of sucrose fatty acid ester, 0.2˜1.5 wt part of potassium pyrophosphate, 0.05˜0.3 wt part of glacial acetic acid, 1.0˜5.0 wt part of iota-carageenan and 0.1˜2.5 wt part of agar.

### BRIEF DESCRIPTION OF THE DRAWINGS

FIG. 1 shows a schematic view indicating process and apparatus for detecting mechanical film strength of cellulose hard capsule of present invention. The film strength is measured by dropping and pressing with the hammer at 4 mm depth from surface of capsule.

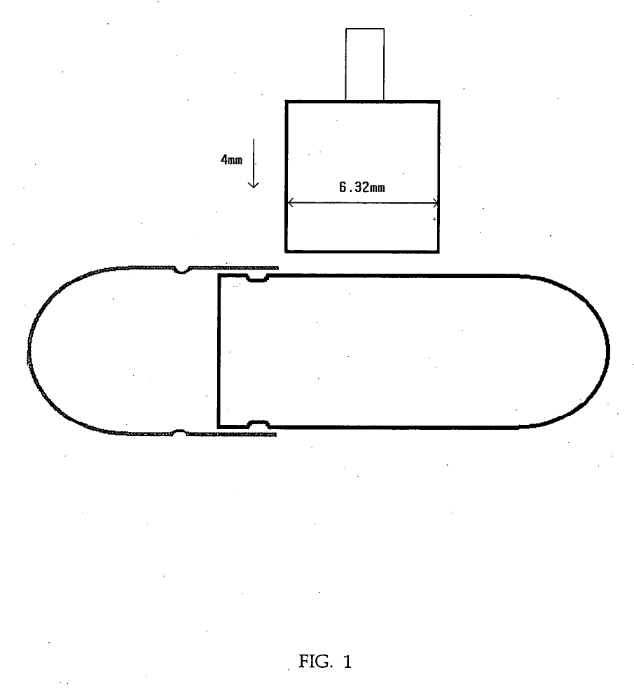

### DETAILED DESCRIPTION OF THE INVENTION

For preparing cellulose hard capsule, physical gelling method or chemical gelling method can be employed after dipping the molding pin in solubilized cellulose solution.

Physical gelling method uses heated (more than 67° C.) molding pin for gelling the solubilized cellulose solution. On the other hand, chemical gelling method requires addition of polysaccharide gelling agent to solubilized cellulose solution.

Even though the cellulose film strength prepared by physical gelling method is higher than that of chemical gelling method, the cellulose film strength by physical gelling method is still lower than that of gelatin film. Table 1 shows the data of cellulose film strength according to gelling method in comparison to gelatin film strength.

TABLE 1

| | | | Film strength (g) | | |
|---|---|---|---|---|---|
| Items | | | Max. | Mean | Min. |
| Gelatin hard capsule | | | 561 | 468 | 417 |
| Cellulose | Physical gelling | | 448 | 356 | 290 |
| hard | method | | | | |
| capsule | Chemical | Pectin | 292 | 234 | 200 |
| | gellling | Gellan gum | 343 | 300 | 276 |
| | method | Carageenan | 382 | 338 | 280 |

The mechanical film strength is measured by Texture Analyser (Model TA 1000). Dropping speed of measuring hammer is 0.5 mm/sec and the depth of measurement of mechanical film strength is 4 mm from surface of capsule. The testing capsule is laid on Texture Analyser and the film strength is measured at 4 mm pressing depth by dropping the hammer.

As shown in Table 1, mechanical film strength of cellulose hard capsule prepared by physical gelling method is higher than that of chemical gelling method. Nevertheless, the most of capsule manufacturer prefers the chemical gelling method, because chemical gelling method does not require the reform of molding pin or other capsule manufacturing machine, whereas physical gelling method requires additional cost for reform of molding pin and other capsule manufacturing machine.

However, cellulose hard capsule prepared by chemical gelling method has some above mentioned handicaps because of low mechanical film strength compared to that of gelatin hard capsule.

Carageenan can be classified into 3 types; kappa-carageenan, iota-carageenan and ramda-carageenan. Among them, kappa-carageenan and iota-carageenan have double helix structure connecting 2 moleculars through chain in 3-dimension structure, which results in the formation of gel to be used for manufacturing capsule. However, ramda-carageenan does not have double helix structure, which cannot be used for manufacturing capsule.

Especially, kappa-carageenan has been used as gelatinizing agent, because strong gel is formed in combination with potassium ion such as potassium chloride. However, iota-carageenan can be a better gelatinizing agent when it is mixed with compatible auxiliary for gelation, because double helix structure of iota-carageenan is more compatible for forming gel.

To search the compatibility of iota-carageenan for forming gel, various mixed solutions of iota-carageenan and other polysaccharide are adopted as gelatinizing agent for sample cellulose capsule for testing the enhancement of mechanical film strength.

Other polysaccharide employed for this test can be one or more selected from agar gum, guar gum, locust bean gum, gellan gum, xanthan gum, pectin, starch, pulluran and agar. Mixed solution of iota-carageenan and agar shows the best combination as gelatinizing agent with the enhancement of mechanical film strength of cellulose hard capsule.

Through the test, we also find that the use of mixed solution of iota-carageenan and agar shows the better mechanical film strength of cellulose hard capsule compared to the use of kappa-carageenan alone or combination.

We also test the selection of auxiliary for gelation of iota-carageenan among potassium phosphate monobasic, potassium phosphate dibasic, potassium gluconate, potassium citrate, potassium carbonate and potassium pyrophosphate. Potassium pyrophosphate shows the best auxiliary for gelation of iota-carageenan.

Further, glacial acetic acid has been required for formation of gel to adjust pH of cellulose aqueous solution to avoid alkaline by adding potassium pyrophosphate. Adding glacial acetic acid adjusts pH of cellulose aqueous solution to be about pH 6.

On the other hand, sucrose fatty acid ester has been also required as emulsifier for excellent film distribution as well as film strength.

The present invention will be more specifically explained by the following examples. However, it should be understood that the examples are intended to illustrate but not in any manner to limit the scope of the present invention.

### EXAMPLES

### (Preparation Examples 1˜3) Preparation of Cellulose Hard Capsule

Hydroxypropylmethylcellulose (HPMC) is added to purified water (about 80° C.). Then, the mixture is stirred and dispersed. Sucrose fatty acid ester, potassium pyrophosphate and glacial acetic acid are also added to solubilized cellulose solution. Then, mixed solution of iota-carageenan and agar is added to resulting admixture and stirred at 60 rpm for 2 hours. After stirring, the mixed cellulose solution (about 80° C.) is cooled to about 4 5° C. Then, the mixed cellulose solution is heated again until 58° C. for molding. Finally, cellulose hard capsule is formed from capsule manufacturing machine.

According to above preparation method, cellulose hard capsules have been prepared with following composition contents as shown in Table 2. The content and ratio of iota-carageenan and agar have been changed to same content of cellulose base material. On the other hand, the contents of sucrose fatty acid ester, potassium pyrophosphate and glacial acetic acid have corresponded to be all same in these preparation examples.

TABLE 2

| Items | Pre. Ex. 1 | Pre. Ex. 2 | Pre. Ex. 3 |
|---|---|---|---|
| HPMC | 94.751 | 94.751 | 94.751 |
| Sucrose fatty acid | 0.9475 | 0.9475 | 0.9475 |
| ester | | | |
| Potassium | 0.4737 | 0.4737 | 0.4737 |
| pyrophosphate | | | |
| Glacial acetic acid | 0.1421 | 0.1421 | 0.1421 |
| Iota-carageenan | 3.1321 | 3.3163 | 3.5006 |
| Agar | 0.5528 | 0.3686 | 0.1843 |

### (Example 1) Measurement of Mechanical Film Strength of Cellulose Hard Capsule

Mechanical film strength of cellulose hard capsule prepared in Preparation Examples 1˜3 and control have been measured using #0 size capsule. Cellulose hard capsule used for control has been prepared according to the method disclosed in Example 1 of U.S. Pat. No. 5,431,917. The results of mechanical film strength are shown in Table 3.

TABLE 3
Comparison of mechanical film strength (n:100)

| | | | | | | Gelatin |
|---|---|---|---|---|---|---|
| | | Pre. | Pre. | Pre. | | hard |
| Items | | Ex. 1 | Ex. 2 | Ex. 3 | Control | capsule |
| Mechanical | Max. | 468 | 452 | 406 | 396 | 561 |
| film | Mean | 413 | 405 | 350 | 344 | 468 |
| strength | Min. | 380 | 364 | 304 | 297 | 417 |
| (g) | | | | | | |

* The method for measuring mechanical film strength is same as the method used in Table 1.

As shown in Table 3, the mechanical film strength prepared in Preparation Examples 1 and 2 shows better mechanical film strength prepared by physical gelling method. It means that mechanical film strength increases according to increase of agar component in the mixture of iota-carageenan and agar. Further, mechanical film strength prepared in Preparation Examples 1˜3 shows better mechanical film strength than that of control. It means that iota-carageenan and agar is better gelatinizing agent than kappa-carageenan alone.

### (Example 2) Measurement of Film Transparency of Cellulose Hard Capsule

Film transparency of cellulose hard capsule prepared by Preparation Examples 1˜3 and control has been measured using UV-visible ray spectrophotometer at 570 nm. Cellulose hard capsule used for control has been prepared according to the method disclosed in U.S. Pat. No. 6,410,050 B1. Testing samples have been prepared in a length 1 cm and a width 1 cm from body part of cellulose capsule. The results of film transparency are shown in Table 4.

TABLE 4
Comparison of film transparency

| Item | Pre. Ex. 1 | Pre. Ex. 2 | Pre. Ex. 3 | Control |
|---|---|---|---|---|
| Transparency | 42% | 50% | 51% | 48% |
| (%) | | | | |

As shown in Table 4, the film transparency prepared by Preparation Examples 2˜3 is better than that of control. However, the film strength prepared by Preparation Example 3 is not enough to be commercially used. Therefore, cellulose hard capsule prepared by Preparation Example 2 shows the better both film strength and film transparency.

### (Example 3) Preparation of Cellulose Hard Capsule of Present Invention

19.5 Kg of hydroxypropylmethylcellulose (HPMC) (concentration: 19.5%) is added to 79.915 L of purified water (about 80° C.). Then, the mixture is stirred and dispersed. 195 g of sucrose fatty acid ester (concentration: 0.195%), 97.5 g of potassium pyrophosphate (concentration: 0.0975%) and 29.25 g of glacial acetic acid (concentration: 0.0292%) are also added to solubilized cellulose solution. Then, mixed solution of 682.5 g of iota-carageenan (concentration: 0.6825%) and 75.855 g of agar (concentration: 0.0758%) is added to resulting admixture and stirred at 60 rpm for 2 hours. After stirring, the mixed cellulose solution (about 80° C.) is cooled to about 4 5° C. Then, the mixed cellulose solution is heated again until 58° C. for molding. Finally, cellulose hard capsule is formed from capsule manufacturing machine.

### (Example 4) Filling Up Property of Cellulose Hard Capsule of Present Invention

According to the method disclosed in Example 3, #0 size transparency hard capsule has been prepared. As control, the cellulose hard capsule prepared by the method disclosed in U.S. Pat. No. 6,410,050 B1 has been employed. The filling up property of cellulose hard capsule and control is shown in Table 5.

TABLE 5
Comparison of filling up property

| Type of filling up machine | EXC-100F | |
|---|---|---|
| Filling up velosity | 50,000/hr | 100,000/hr |
| Pressure | 20 cmHg | 20 cmHg |
| Amount | 500,000 EA | 500,000 EA |

| | | | Present | | Present |
|---|---|---|---|---|---|
| | Items | Control | Invention | Control | Invention |
| Result | Telescope | 15 | 1 | 20 | 3 |
| | Mashed badness | 8 | 0 | 10 | 1 |

As shown in Table 5, the cellulose hard capsule prepared in Example 3 shows better filling up property by reducing the telescope and mashed badness in filling procedure. This means that film strength of cellulose hard capsule of present invention is excellent to be commercially used.

### (Example 5) Printing Property of Cellulose Hard Capsule of Present Invention

According to the method disclosed in Example 3, #0 size transparency hard capsule has been prepared. As control, the cellulose hard capsule prepared by the method disclosed in U.S. Pat. No. 6,410,050 B1 has been employed. The printing property of cellulose hard capsule and control is shown in Table 6.

TABLE 6
Comparison of printing property

| | |
|---|---|
| Type of printing machine | EXC-100R (Spin printer) |
| Printing velosity | 100,000/hr |
| Amount | 500,000 EA |

| | Control | Present Invention |
|---|---|---|
| Telescope or | 10 EA | 1 EA |
| Mashed badness | | |

As shown in Table 6, the cellulose hard capsule prepared in Example 3 shows better printing property by reducing the telescope and mashed badness in printing procedure. This means that film strength of cellulose hard capsule of present invention is excellent to be commercially used.

### (Example 6) Roundness of Cellulose Hard Capsule of Present Invention

According to the method disclosed in Example 3, #0 size transparency hard capsule has been prepared. As control, the cellulose hard capsule prepared by the method disclosed in U.S. Pat. No. 6,410,050 B1 has been employed. The roundness is measured using profile projector after dividing the capsule into cap and body. The roundness of cellulose hard capsule and control is shown in Table 7.

TABLE 7
Comparison of roundness (n:50)

| | |
|---|---|
| Control | Present Invention |

| Item | Cap | Body | Cap | Body |
|---|---|---|---|---|
| Roundness | 98.5% | 98.3% | 99.3% | 99.1% |
| (%) | | | | |

As shown in Table 7, the cellulose hard capsule prepared in Example 3 shows better roundness. This means that film strength of cellulose hard capsule of present invention is excellent to be commercially used.

As shown in Examples, the filling up property, printing property and roundness of cellulose hard capsule in present invention are all excellent. This means that the film strength of cellulose hard capsule in present invention is fully enhanced. Of course, the film strength of cellulose hard capsule in present invention is better than any of previously known capsule disclosed in U.S. Pat. No. 5,431,917 and U.S. Pat. No. 6,410,050 B1.

What is claimed is:

1. A process for preparing a cellulose capsule enhancing mechanical film strength comprising the steps of:
i) preparing an aqueous solution of solubilized cellulose;
ii) adding sucrose fatty acid ester, potassium pyrophosphate and glacial acetic acid to an aqueous solution of solubilized cellulose;
iii) adding a mixed solution of iota-carageenan and agar to the resulting admixutre; and
iv) allowing obtained product to stand, adjusting its viscosity and forming a capsule from it.

2. The process for preparing a cellulose capsule enhancing mechanical film strength according to claim 1, comprising the steps of:
preparing 100 wt part of an aqueous solution containing 18˜21 wt part of solubilized cellulose;
ii) adding 0.1˜0.5 wt part of sucrose fatty acid ester, 0.05˜0.3 wt part of potassium pyrophosphate and 0.01˜0.2 wt part of glacial acetic acid to 100 wt part of an aqueous solution of solubilized cellulose;
iii) adding a mixed solution of 0.1˜1.0 wt part of iota-carageenan and 0.02˜0.5 wt part of agar to the resulting admixutre; and
iv) allowing obtained product to stand, adjusting its viscosity and forming a capsule from it.

3. The process for preparing a cellulose capsule enhancing mechanical film strength according to claim 1, wherein the weight ratio of iota-carageenan and agar is 3.5˜4.5:1.

4. The process for preparing a cellulose capsule enhancing mechanical film strength according to claim 1, wherein said cellulose is hydroxypropylmethylcellulose (HPMC).

5. The process for preparing a cellulose capsule enhancing mechanical film strength according to claim 1, wherein said glacial acetic acid is added to neutralize the pH of an alkaline solution of solubilized cellulose.

6. A cellulose hard capsule prepared by the preparation method of claim 1.

7. The cellulose hard capsule according to claim 6, wherein said cellulose hard capsule comprises 100 wt part of cellulose, 0.5˜2.5 wt part of sucrose fatty acid ester, 0.2˜1.5 wt part of potassium pyrophosphate, 0.05˜0.3 wt part of glacial acetic acid, 1.0˜5.0 wt part of iota-carageenan and 0.1˜2.5 wt part of agar.
